(19) United States
(12) Patent Application Publication
(10) Pub. No.: US 2019/0388007 A1
(43) Pub. Date: Dec. 26, 2019

(54) EXHALED AIR MEASUREMENT DEVICE

(71) Applicant: PHC HOLDINGS CORPORATION, Tokyo (JP)

(72) Inventors: Kenji OKI, Ehime (JP); Takanori KONISHI, Ehime (JP); Takashi KIMURA, Ehime (JP)

(21) Appl. No.: 16/471,503

(22) PCT Filed: Mar. 22, 2018
(86) PCT No.: PCT/JP2018/011483

§ 371 Date: Jun. 19, 2019

(30) Foreign Application Priority Data
Mar. 23, 2017 (JP) 2017-057132

(51) Int. Cl. A61B 5/097; A61B 5/083; A61B 5/00

(52) U.S. Cl. CPC A61B 5/097; A61B 5/742; A61B 5/083

(57) ABSTRACT

An exhalation measurement device includes: a chamber to retain exhalation; a reference gas generator 17 to generate a reference gas; a measurement device 12 to measure concentrations of exhalation being retained in the chamber and a specific gas within the reference gas; a gas transporter 18 including a pump 11 to selectively transport the exhalation being retained in the chamber and the reference gas generated by the reference gas generator to the measurement device; and a control circuit 50 to control an operation of the gas transporter. The reference gas generator 17 includes: a case 19 having an intake port and an exhaust port, and an aeration path connecting the intake port and the exhaust port; a first filter 23 having a first aperture and a second aperture respectively on the intake port side and the exhaust port side in the aeration path within the case 19, the first filter 23 being disposed in the aeration path to adsorb the specific gas; and a supply tube 29 having a third aperture and a fourth aperture, the fourth aperture being connected to the intake port of the case. A distance from the third aperture of the supply tube to the first aperture of the first filter is longer than a distance between the first aperture and the second aperture of the first filter.

### TECHNICAL FIELD

The present disclosure relates to an exhalation measurement device to be used when e.g. detecting asthma or checking pulmonary function.

### BACKGROUND ART

Exhalation measurement devices for detecting a specific gas that is contained in the exhalation of a subject, for use in disease diagnosis or pulmonary function evaluation, have been put into practical use. For example, Patent Document 1 discloses a diagnostic gas analyzer that detects nitrogen monoxide (NO) within exhalation. Patent Document 1 states that the amount of nitrogen monoxide within exhalation can be used as an index of pneumonia diagnosis, or put to clinical use for asthma patients.

### CITATION LIST

### Patent Literature

Japanese National Phase PCT Laid-Open Publication No. 2005-538819

### SUMMARY OF INVENTION

### Technical Problem

Since the content of any specific gas, e.g., nitrogen monoxide, within exhalation would be very small, an exhalation measurement device is required to be able to stably detect the amount of specific gas while maintaining high accuracy. The present disclosure provides an exhalation measurement device which is capable of stably detecting the amount of specific gas while maintaining high accuracy.

### Solution to Problem

An exhalation measurement device according to one implementation of the present disclosure comprises: a chamber to retain exhalation; a reference gas generator to generate a reference gas; and a measurement device to measure concentrations, respectively, of exhalation being retained in the chamber and a specific gas within the reference gas, the reference gas generator including: a case having an intake port and an exhaust port, and an aeration path connecting the intake port and the exhaust port; a first filter having a first aperture and a second aperture respectively on the intake port side and the exhaust port side in the aeration path within the case, the first filter being disposed in the aeration path to adsorb the specific gas; and a supply tube having a third aperture and a fourth aperture, the fourth aperture being connected to the intake port of the case, wherein a distance from the third aperture of the supply tube to the first aperture of the first filter is longer than a distance between the first aperture and the second aperture of the first filter.

### Advantageous Effects of Invention

With an exhalation measurement device according to the present disclosure, it is possible to stably detect the amount of specific gas while maintaining high accuracy.

### BRIEF DESCRIPTION OF DRAWINGS

FIG. 1 is a perspective view showing an exemplary embodiment of an exhalation measurement device according to the present disclosure.

FIG. 2 shows an example control block diagram of the exhalation measurement device.

FIG. 3 is a perspective view showing an example of the interior of a main body of the exhalation measurement device.

FIG. 4 is a cross-sectional view showing an example of a handle section.

FIG. 5A is a schematic diagram showing an example of an adjuster.

FIG. 5B is a schematic diagram showing an example where a gauge pressure sensor is used as a pressure sensor.

FIG. 5C is a schematic diagram showing an example where a differential pressure sensor is used as a pressure sensor.

FIG. 5D is a schematic diagram showing an example where an atmospheric pressure sensor is used as a pressure sensor.

FIG. 6 is a cross-sectional view showing an example of a reference gas generator.

FIG. 7 is a schematic diagram showing an example of a switching device.

FIG. 8A is a schematic diagram showing an example of a flow rate detector.

FIG. 8B is a schematic diagram showing an example of a flow rate detector.

FIG. 9 is a flowchart showing an exemplary operation of the exhalation measurement device.

FIG. 10A is a graph showing a result of examining the characteristics of reference gas generators.

FIG. 10B is a graph showing a result of examining the characteristics of reference gas generators.

### DESCRIPTION OF EMBODIMENTS

The inventors have sought for a method which can stably measure a specific gas content within exhalation while maintaining high accuracy. In order to enhance the accuracy of measurement, the amount of specific gas contained in the inhalation of a subject should preferably be reduced as much as possible in advance. Moreover, a reference gas which was generated under conditions resembling inhalation may be provided, and the specific gas content within exhalation may be measured against this reference gas. In order to generate such a reference gas, the specific gas within the reference gas should also preferably be reduced as much as possible.

In order to reduce the amount of specific gas within air, use of a reference gas generator may be possible, such that the reference gas generator includes a filter which is filled with an adsorbent that adsorbs the specific gas. The inventors have tried producing an exhalation measurement device equipped with such a reference gas generator, and measuring the amount of specific gas within exhalation under various conditions, thereby finding that the reference gas generator may deteriorate early on in some cases. In looking for the causes thereof, they found that at a medical institution, etc., where the exhalation measurement device is used, other gases may exist in the surrounding environment which induce early deterioration of the adsorbent that adsorbs the specific gas. For example, in a medical institution or the like, ethyl alcohol may be used for disinfection of medical equipment, or disinfection of the patient's skin, etc. Hereinafter, ethyl alcohol will simply be referred to as alcohol in the present specification. If any such alcohol exists in the environment in which the exhalation measurement device is stored or in the environment of use, the adsorbent will adsorb the alcohol, and be degraded with respect to its ability to adsorb the specific gas. Especially when the exhalation measurement device is stored in its dedicated case or the like after alcohol disinfection, alcohol may vaporize within the dedicated case, so that a temporarily high concentration of alcohol may exist. In such cases, the high concentration of alcohol may be adsorbed by the adsorbent, thereby significantly deteriorating the adsorbent's ability to adsorb the specific gas.

In view of such problems, the inventors have arrived at a novel exhalation measurement device. In summary, an exhalation measurement device according to the present disclosure is as follows.

### [Item 1]

An exhalation measurement device comprising:

a chamber to retain exhalation;

a reference gas generator to generate a reference gas; and

a measurement device to measure concentrations, respectively, of exhalation being retained in the chamber and a specific gas within the reference gas,

the reference gas generator including:

a case having an intake port and an exhaust port, and an aeration path connecting the intake port and the exhaust port;

a first filter having a first aperture and a second aperture respectively on the intake port side and the exhaust port side in the aeration path within the case, the first filter being disposed in the aeration path to adsorb the specific gas; and

a supply tube having a third aperture and a fourth aperture, the fourth aperture being connected to the intake port of the case, wherein

a distance from the third aperture of the supply tube to the first aperture of the first filter is longer than a distance between the first aperture and the second aperture of the first filter.

### [Item 2]

The exhalation measurement device of claim 1, wherein a distance between the third aperture and the fourth aperture of the supply tube is longer than the distance between the first aperture and the second aperture of the first filter.

### [Item 3]

The exhalation measurement device of claim 1, wherein the distance between the third aperture and the fourth aperture of the supply tube is 50 mm or more.

### [Item 4]

The exhalation measurement device of claim 1, wherein the distance between the third aperture and the fourth aperture of the supply tube is 100 mm or more.

### [Item 5]

The exhalation measurement device of claim 1, wherein a channel of the supply tube has a cross section which is smaller than a cross section of the first aperture that is perpendicular to a straight line connecting the first filter and the second aperture.

### [Item 6]

The exhalation measurement device of claim 1, wherein the supply tube has an inner diameter of 5 mm or less.

### [Item 7]

The exhalation measurement device of claim 1, wherein the supply tube is composed of a resin.

### [Item 8]

The exhalation measurement device of claim 7, wherein the resin is a fluoroplastic.

### [Item 9]

The exhalation measurement device of claim 1, wherein the reference gas generator includes a check valve disposed between the intake port of the case and the first aperture.

### [Item 10]

The exhalation measurement device of claim 1, comprising:

a gas transporter including a pump to selectively transport the exhalation being retained in the chamber or the reference gas generated by the reference gas generator to the measurement device; and

a control circuit to control an operation of the gas transporter.

### [Item 11]

The exhalation measurement device of claim 10, further comprising an exhalation generation unit including:

an aperture through which exhalation and inhalation pass,

an inhalation path having a suction hole and connecting the aperture with the suction hole,

a second filter to adsorb the specific gas, the second filter being located in the inhalation path,

an exhalation path having a discharge hole and connecting the aperture with the discharge hole, wherein,

exhalation which is sent from the discharge hole is introduced into the chamber.

### [Item 12]

The exhalation measurement device of claim 11, further comprising a main housing which accommodates the chamber, the reference gas generator, the measurement device, the gas transporter, and the control circuit, wherein

the exhalation generation unit is mounted to the main housing.

### [Item 13]

The exhalation measurement device of claim 11, further comprising a handle section including a sub-housing which is grippable by a subject, wherein

the exhalation generation unit is accommodated in the sub-housing.

### [Item 14]

The exhalation measurement device of claim 10, further comprising:

an introduction path having an inlet through which the exhalation is introduced, the introduction path being connected to the chamber;

a flow rate adjuster to adjust a flow rate of the exhalation flowing in the introduction path; and a pressure sensor to measure a pressure in the introduction path, wherein,

the control circuit controls the flow rate adjuster based on an output from the pressure sensor.

### [Item 15]

The exhalation measurement device of claim 10, wherein,

the gas transporter further includes

a switching device to switch between a first path connected to the chamber and a second path connected to the pump, and allow the first path or the second path to connect to a third path that is connected to the pump, and

a flow rate detector to measure a flow rate of the exhalation or the reference gas flowing in the third path; and

the control circuit controls the switching device, and controls the pump based on an output from the flow rate detector.

Hereinafter, with reference to the drawings, an embodiment of an exhalation measurement device according to the present disclosure will be described. An exhalation measurement device according to the present disclosure may measure the concentration of nitrogen monoxide within exhalation, for example. In the present embodiment, inhalation means the air that is sucked into a subject who breathes in, or the air that moves during a sucking motion of the subject. On the other hand, exhalation means a gaseous body that is discharged out of the subject as the subject breathes out.

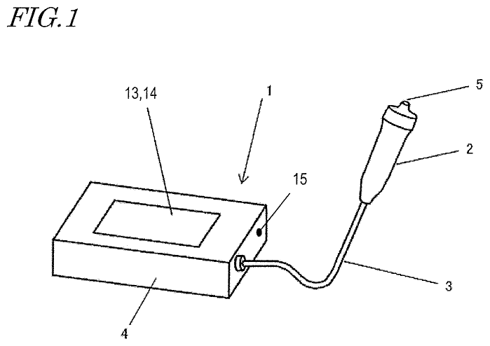

FIG. 1 is a perspective view showing the appearance of an exhalation measurement device 1. The exhalation measurement device 1 includes, for example, a handle section 2, a main body 4, and a handle tube 3 that connects the handle section 2 and the main body 4.

A subject holds the handle section 2 in a hand, and after breathing out, places an orifice 5 of the handle section 2 against his or her mouth, and breathes in while in this state. Thereafter, as he or she breathes out, exhalation flows from the orifice 5 into the handle section 2, whereby the exhalation is introduced into the main body 4 via the handle tube 3. The main body 4 measures the nitrogen monoxide concentration within the exhalation, and displays the result of measurement on a display device 14 of the main body 4.

### [Structure of the Exhalation Measurement Device 1]

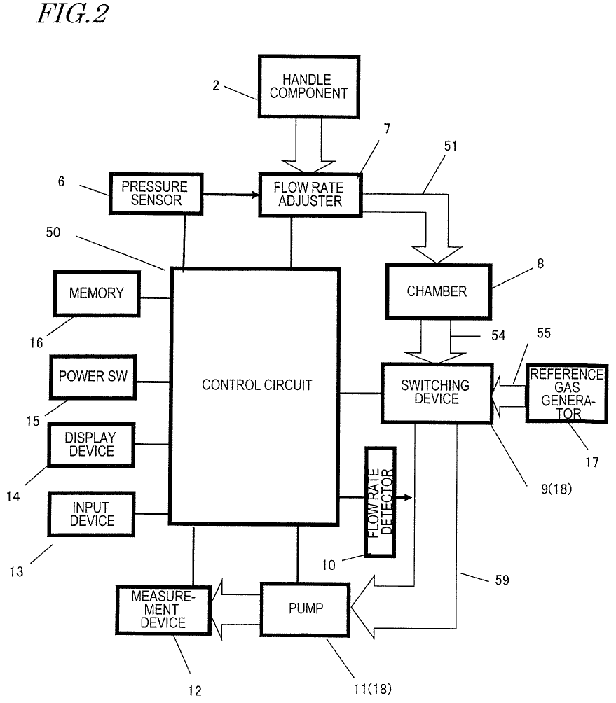

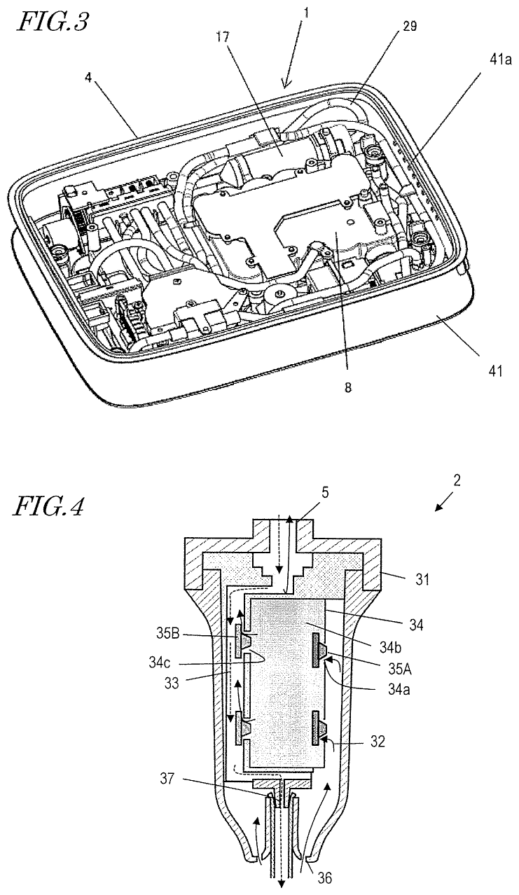

FIG. 2 is a control block diagram of the main body 4. In FIG. 2, thick arrows schematically show channels through which a gas moves, as well as directions of its movement. FIG. 3 is a perspective view showing the interior of the main body 4. Component elements of the main body 4 are accommodated in a main housing 41. The main body 4 includes: a pressure sensor 6; a flow rate adjuster 7; a chamber 8; a measurement device 12; an input device 13; the display device 14; a power switch 15; a memory 16; a reference gas generator 17; a gas transporter 18 including a switching device 9, a flow rate detector 10, and a pump 11; and a control circuit (control device) 50.

The exhalation measurement device 1 is controlled by the control circuit 50. The control circuit 50 includes electronic parts with processing circuitry for performing information processing, such as a CPU provided in the exhalation measurement device 1; it is also called a microcomputer. By executing a computer program that is loaded to the memory 16, the control circuit 50 sends instructions to other component elements, in accordance with the procedure of the computer program. Receiving such instructions, the respective component elements operate as will be described in the present specification. The procedure of the computer program will be described later.

The input device 13 is a device with which a person, e.g., a subject, who operates the exhalation measurement device 1 inputs information to the control circuit 50, including for example a touch screen panel that is integrally provided within the display device 14. The power switch 15 is provided in the main housing 41, for example.

The memory to which the computer program is loaded may be volatile or non-volatile. The volatile memory (RAM) is a RAM that cannot retain its stored information while not being powered. For example, a dynamic random access memory (DRAM) is a typical volatile RAM. A non-volatile RAM is a RAM that is capable of retaining information without being powered. For instance, magnetoresistive RAMs (MRAM), resistive random access memories (ReRAM), and ferroelectric resistive random access memories (FeRAM) are examples of non-volatile RAMs. In the present embodiment, a non-volatile RAM is preferably adopted. A volatile RAM and a non-volatile RAM are both examples of non-transitory and computer-readable storage media. Moreover, a magnetic storage medium such as a hard disk, and an optical storage medium such as an optical disc, are also examples of non-transitory and computer-readable storage media. That is, a computer program according to the present disclosure may be recorded on any of various non-transitory computer-readable media, other than any medium such as the atmospheric air (i.e., a transitory medium), that transmits the computer program as a radio wave signal. Hereinafter, each component element will be described in detail.

(Handle Section 2)

The handle section 2 is an exhalation generation unit that causes a subject's exhalation to be generated. In the present embodiment, the exhalation generation unit is accommodated, as the handle section 2, in a separate housing from the main body 4. FIG. 4 shows a schematic cross section of an example of the handle section 2. The handle section 2 includes a sub-housing 31, an inhalation path 32, an exhalation path 33, and a second filter 34. The sub-housing 31 has an outer shape that allows the subject to grip it, for example. Specifically, the sub-housing 31 may have a substantially cylindrical outer shape that can be gripped with the palm of a hand, with a suction hole 36 and a discharge hole 37 being provided at one end of the cylindrical sub-housing 31, for example. At the other end of the cylindrical sub-housing 31, the orifice 5, through which inhalation and exhalation of the subject pass, is provided. The inhalation path 32 connects between the orifice 5 and the suction hole 36, such that the second filter 34 is positioned in the inhalation path 32. The exhalation path 33 connects between the orifice 5 and the discharge hole 37. The inhalation path 32 and the exhalation path 33 are partitioned by the piece that composes the sub-housing 31 or the like, for example.

As the subject breathes in, while holding against his or her mouth the surface of the sub-housing 31 in which the orifice 5 is made, inhalation (air) is introduced into the inhalation path 32.

The second filter 34 inserted in the inhalation path 32 has apertures 34a and 34c and one-way valves 35A and 35B. When the handle section 2 is not being used by the subject, the one-way valves 35A and 35B close the apertures 34a and 34c, respectively.

As the subject breathes in, the one-way valves 35A and 35B open the apertures 34a and 35c, respectively, and the inhalation within the inhalation path 32 is introduced through the aperture(s) 34a into the second filter 34. The second filter 34, which contains an adsorbent 34b that adsorbs a specific gas, adsorbs the specific gas within the inhalation as the inhalation passes through the adsorbent 34b. In the present embodiment, the specific gas is nitrogen monoxide, so that nitrogen monoxide within the inhalation is adsorbed by the adsorbent 34b. The inhalation, from which nitrogen monoxide has been removed to a predetermined concentration or less, is discharged out of the second filter through the aperture(s) 34c equipped with the one-way valve(s) 35, and is sucked into the body of the subject through the orifice 5.

As the subject breathes in and then breathes out, his or her exhalation is introduced through the orifice 5 into the exhalation path 33. In the present embodiment, inhalation and exhalation pass through the orifice 5. This means that the inhalation path 32 and the exhalation path 33 at least have a common portion. In the present embodiment, the region extending from a region of the inhalation path 32 that is in contact with the one-way valve(s) 35 to the orifice 5 defines a path that is common to the exhalation path 33. In other words, the exhalation which is introduced into the exhalation path 33 is in contact with the one-way valve(s) 35. However, owing to a pressure difference between the exhalation path 33 and the interior of the second filter 34, the one-way valve(s) 35 closes the aperture(s) 34c so that the exhalation does not flow into the second filter 34. The exhalation passing through the exhalation path 33 is released out of the handle section 2 through the discharge hole 37. The discharge hole 37 has the handle tube 3 connected thereto, such that exhalation is introduced into the main body 4 through the handle tube 3.

Since the handle section 2 constitutes a separate piece from the main body 4, when the subject carries out an exhalation measurement, he or she may hold only the handle section 2 in order to generate exhalation. Since the subject does not need to hold the main body 4, the subject's burden during examination is reduced. Moreover, excellent operability is provided because the position of the main body may be relatively freely determined, such that the information that is displayed by the display device 14 of the main body 4 will be easy to see. Moreover, in the present embodiment, nitrogen monoxide is removed from the air (i.e., inhalation) that is sucked by the subject in generating exhalation. By removing as much nitrogen monoxide, which is the target of measurement, from the inhalation as possible, influences of the measurement environment on the measurement can be reduced.

In the event that the second filter 34 deteriorates in terms of adsorption performance, the handle section 2 may be constructed so that the entire handle section 2 can be exchanged, or that only the second filter 34 can be exchanged.

In the present embodiment, the exhalation generation unit is composed of the handle section 2 including the sub-housing 31, which is separate from the main body 4. However, for example, the exhalation generation unit may be integral with the main body 4, or composed as a mouthpiece which is detachably mounted to and supported on the main body 4. In this case, the subject may carry out measurement by holding against his or her mouth the mouthpiece mounted on the main body 4, while supporting the main body 4 with a hand.

(Chamber 8)

The chamber 8 has a space for temporarily retaining the exhalation that is generated by the handle section 2. The exhalation which has been introduced from the handle tube 3 to the main body 4 is transported via an introduction path 51 to the chamber 8, owing to the pressure of the exhalation that is put out from the subject. The space in the chamber 8 has a sufficient volumetric capacity for measuring the concentration of the specific gas. Prior to the measurement, the chamber 8, the handle section 2, the handle tube 3, and the introduction path 51 are filled with the same air as the external environment. Therefore, the chamber 8 having a discharge port, the air filling these portions is first discharged through the discharge port from the chamber 8 while the subject keeps breathing out for a certain period of time, and thereafter the exhalation fills the chamber 8.

(Flow Rate Adjuster 7)

When generating exhalation, the concentration of the specific gas within the exhalation may vary depending on the intensity (pressure) and flow rate with which the subject breathes out. In order to carry out measurement under conditions that are as constant as possible, preferably a structure for adjusting the flow rate of exhalation is provided in the introduction path 51 leading to the chamber 8, so that exhalation will be generated within a certain range of pressure and flow rate. For this purpose, in the present embodiment, the main body 4 includes the flow rate adjuster 7 and the pressure sensor 6, such that a pressure in the introduction path 51 is detected by the pressure sensor 6, and, based on the result of detection, a flow rate of exhalation flowing in the introduction path 51 is adjusted by the flow rate adjuster 7.

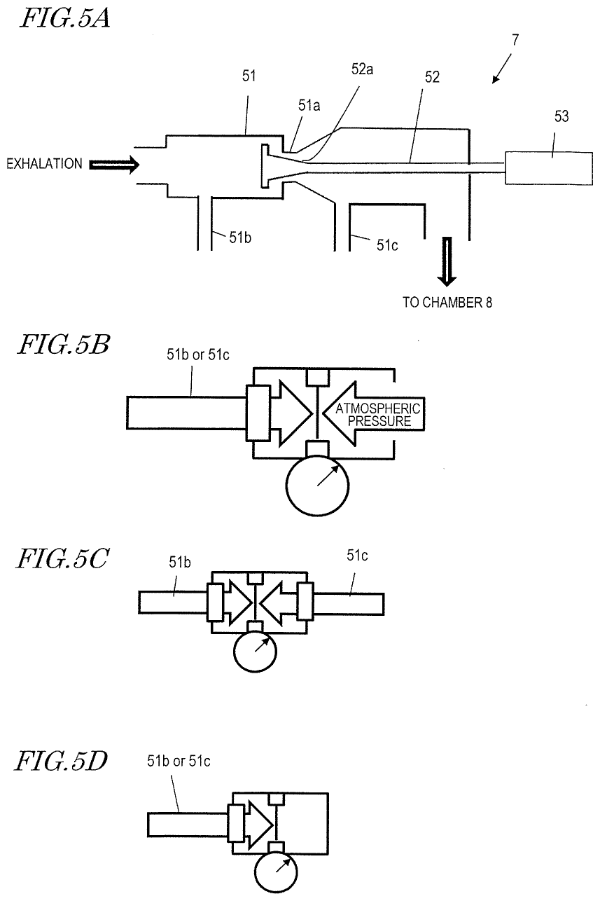

FIG. 5A is a schematic diagram showing an exemplary structure of the flow rate adjuster 7. The flow rate adjuster 7 includes an adjustment valve 52 having a tapered portion 52a, and a driving device 53. An adjustment hole 51a is provided midway in the introduction path 51, and the tapered portion 52a of the adjustment valve 52 is inserted through the aperture of the adjustment hole 51a so that, as the driving device 53 moves the adjustment valve 52, the tapered portion 52a adjusts the size of the aperture, thereby adjusting the flow rate of the exhalation flowing in the introduction path 51. The driving device 53 may be a motor, piezoelectric element, or the like. The flow rate adjuster 7 may be a solenoid valve.

(Pressure Sensor 6)

The pressure sensor 6 detects a pressure in the introduction path 51. Various types of pressure sensors can be used for the pressure sensor. The pressure in the introduction path 51 to be detected by the pressure sensor 6 may be found at: two points, i.e., a port 51b that is located between an inlet through which exhalation is introduced into the main body 4 and the flow rate adjuster 7, and a port 51c that is located between the flow rate adjuster 7 and the chamber 8; or one point, i.e., the port 51b that is located between the inlet through which exhalation is introduced into the main body 4 and the flow rate adjuster 7.

Specifically, the pressure sensor 6 may be a gauge pressure sensor, a differential pressure sensor, an atmospheric pressure sensor, or the like. FIG. 5B schematically shows a construction where pressure is measured by a gauge pressure sensor. The gauge pressure sensor measures a difference in pressure between a space that is connected to the pressure sensor and the atmospheric air. In this case, the pressure sensor 6 may measure a pressure in the introduction path 51 by using the port 51b, or measure pressures by using the port 51b and the port 51c.

FIG. 5C schematically shows a construction where pressure is measured by a differential pressure sensor. The differential pressure sensor measures a pressure difference between two spaces that are connected to the pressure sensor. In this case, the pressure sensor 6 take a measurement at the port 51b and the port 51c.

FIG. 5D schematically shows a construction where pressure is measured by an atmospheric pressure sensor. The atmospheric pressure sensor measures an atmospheric pressure in a space that is connected to the pressure sensor. In this case, the pressure sensor 6 may measure a pressure in the introduction path 51 by using the port 51b, or measure pressures by using the port 51b and the port 51c.

(Reference Gas Generator 17)

The reference gas generator 17 generates a reference gas. The reference gas is preferably the air in an environment which is similar to the inhalation to be sucked in by the subject. This allows conditions other than the specific gas for measurement to be as identical as possible, so that the influence of any conditions other than the specific gas that is exerted on the measurement will be reduced as much as possible. More specifically, it is preferable that: the reference gas generator 17 is capable of generating, as a reference gas, the air which is acquired from a surrounding environment of the exhalation measurement device 1 during measurement but from which the specific gas has been removed.

As shown in FIG. 3, the reference gas generator 17 is disposed inside the main body 4. On the side surface of the main body 4 near the reference gas generator 17, a plurality of external air holes 41a are provided. Through the external air holes 41a, external air is taken into the main body 4 of the measurement device, thus allowing the measurement environment (e.g., temperature and humidity) inside the main body 4 of the measurement device to be identical with the external environment.

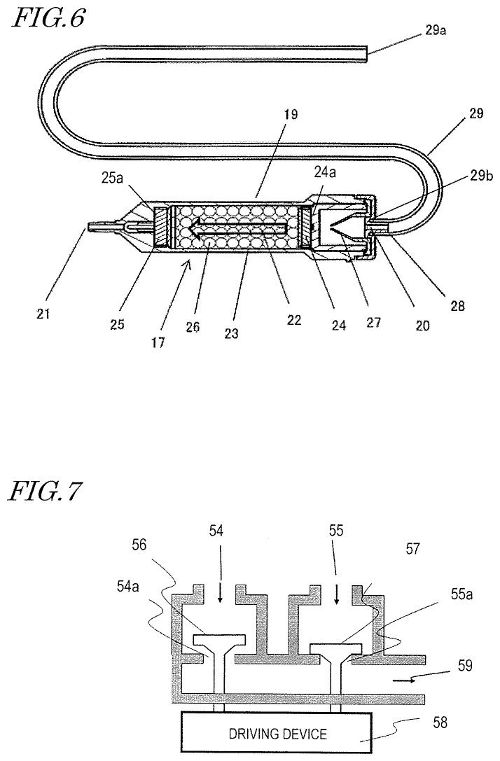

FIG. 6 is a cross-sectional view of the reference gas generator 17. The reference gas generator 17 includes an elongated cylindrical case 19, a first filter 23, and a supply tube 29. At the two ends of its longitudinal direction, the case 19 has an intake port 20 and an exhaust port 21. The intake port 20 and the exhaust port 21 are made with a smaller diameter than the inner diameter of the case 19. The case 19 has an aeration path 22 that connects between the intake port 20 and the exhaust port 21. Around (on the outer surface of) the intake port 20, a cylindrical, outwardly-protruding attachment section 28 is provided.

The first filter 23 is disposed in the aeration path 22. In the present embodiment, the first filter 23 has a cylindrical shape, and includes an air-permeable gasket 24 disposed closer to the intake port 20, an air-permeable gasket 25 disposed closer to the exhaust port 21, and an adsorbent 26 that adsorbs a specific gas, which fills between the gasket 24 and the gasket 25. A surface of the gasket 24 that is on the intake port 20 side will be referred to as a first aperture 24a, and a surface of the gasket 25 that is on the exhaust port 21 side will be referred to as a second aperture 25a.

Inside the case 19 and between the intake port 20 and the first aperture 24a of the gasket 24, a check valve 27 is provided. The check valve 27 is composed of a pair of thin rubber plates, such that its end toward the gasket 24 is opened and closed. Specifically, when air moves from the intake port 20 to the first filter 23, the pair of thin rubber plates of the check valve 27 are deformed so that their interspace expands; this causes the check valve 27 to be open. On the other hand, when air tries to flow from the first filter 23 and the intake port 20, the pair of thin rubber plates will deform so as to abut with each other; this causes the check valve 27 to be closed.

As shown in FIG. 6, when the pressure at the first aperture 24a side and the pressure at the intake port 20 side are equal, i.e., when there is no air movement in the two aforementioned directions, the leading ends of the pair of thin rubber plates of the check valve 27 are spaced apart. In other words, when there is no air movement, the pair of thin rubber plates do not abut with each other. Because of the check valve 27 having this structure, even when the reference gas generator 17 has sucked in highly humid air, the pair of thin rubber plates are restrained from adhering to each other due to humidity.

The supply tube 29 has a third aperture 29a and a fourth aperture 29b; as the attachment section 28 of the case is inserted in the fourth aperture 29b, the fourth aperture 29b becomes connected to the intake port 20. As will be described in detail below, the distance from the third aperture 29a of the supply tube 29 to the first aperture 24a of the first filter 23 is longer than the distance between the first aperture 24a and the second aperture 25a of the first filter. Moreover, the distance between the third aperture 29a and the fourth aperture 29b of the supply tube 29 is longer than the distance between the first aperture 24a and the second aperture 25a of the first filter.

Preferably, the distance between the third aperture 29a of the supply tube 29 and the fourth aperture 29b is 50 mm or more, and more preferably 100 mm or more.

The channel of the supply tube 29 has a cross section which is smaller than a cross section of the first filter that is perpendicular to a straight line connecting between the first aperture 24a and the second aperture 25a, i.e., the longitudinal direction. Preferably, the supply tube 29 has an inner diameter of 5 mm or less.

The supply tube 29 is composed of a resin. Preferably, it is composed of a resin having low gas permeability. For example, the supply tube 29 is preferably composed of a soft urethane, a fluoroplastic such as polytetrafluoroethylene, or the like.

Since the reference gas generator 17 includes the supply tube 29, even when the exhalation measurement device 1 is stored in an environment containing alcohol or the like, deterioration of the first filter 23 is suppressed. This can prolong the period during which highly accurate measurement is possible, thus being able to realize an exhalation measurement device which is capable of stably detecting the amount of specific gas while maintaining high accuracy.

(Gas Transporter 18)

The gas transporter 18 selectively transports the exhalation which is retained in the chamber 8 or the reference gas which is generated by the reference gas generator to the measurement device 12. In the present embodiment, the gas transporter 18 includes the switching device 9, the flow rate detector 10, and the pump 11.

FIG. 7 is a schematic diagram showing an exemplary structure of the switching device 9. The switching device 9 switches between the first path 54 that connects to the chamber 8 and the second path 55 that connects to the reference gas generator 17, and allows it to be connected to the third path 59. Specifically, the switching device 9 includes a valve 56 that is located between the first path 54 and the third path, a valve 57 that is located between the second path 55 and the third path 59, and a driving device 58. Under control of the control circuit 50, the driving device 58 opens or closes the valve 56 and the valve 57. As a result, the switching device 9 selectively connects the first path 54 or the second path 55 to the third path. As the pump 11 sucks in the gas in the selected path, the exhalation or the reference gas is selectively transported to the measurement device 12.

As the switching device 9, various valves to be used in fluid control can be used, e.g., a three-way valve. Alternatively, a plurality of solenoid valves may be used. Furthermore, the gas transporter 18 may include two parallel paths that respectively connect the chamber 8 and reference gas to the measurement device, such that a valve, a pump, and a flow rate detector are provided in each path. In this case, by selectively operating the valves and pumps in the two paths, the exhalation or the reference gas can be selectively transported to the measurement device 12. As the pump 11, commercially available pumps for use with various fluids can be used.

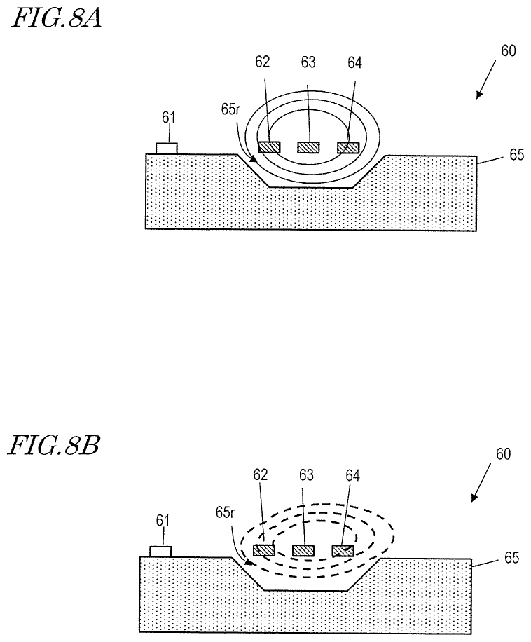

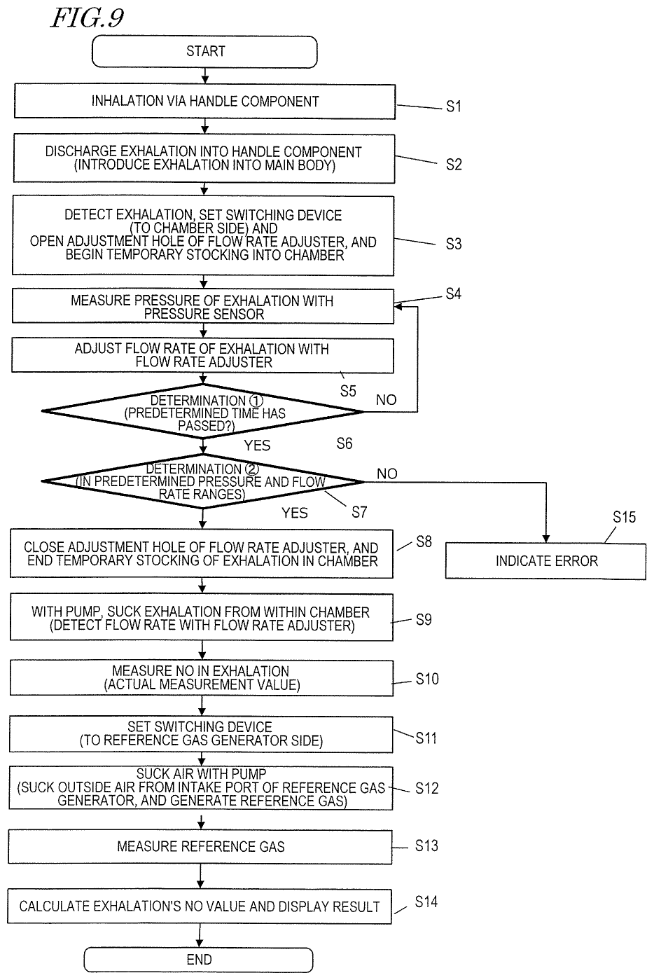

The flow rate detector 10 detects a flow rate of a gas flowing in the third path 59. As the flow rate detector 10, various sensors that detect a flow rate of a fluid can be used. For example, a sensor of a thermal type, a differential-pressure type, etc., can be used. FIG. 8A and FIG. 8B are schematic diagrams showing a thermal-type flow rate detector 60 for use in the flow rate detector 10. The thermal-type flow rate detector includes, for example, an upstream temperature sensor 62 a heater 63, and a downstream temperature sensor 64 that are disposed so as to be spaced apart from the bottom of a recess 65r which is made in a substrate 65 of silicon. Moreover, an ambient temperature sensor 61 is provided on the substrate 65. When an electric current is flowed in the heater 63, with heating of the heater, a temperature distribution that is ascribable to the heating emerges above the recess 65r. As indicated by solid lines in FIG. 8A, when the gaseous body covering the recess 65r is not moving, this temperature distribution is symmetric, so that the upstream temperature sensor 62 and the downstream temperature sensor 64 will detect an equal temperature. However, in an environment where the surrounding gaseous body is moving, as indicated by broken lines in FIG. 8B, the temperature distribution will abound at the downstream side, so that a difference will exist between the temperatures detected by the upstream temperature sensor and the downstream temperature sensor 64. This temperature difference varies with the velocity with which the fluid moves, i.e., flow rate. The detected flow rate may be corrected based on the temperature in the surroundings as obtained by the ambient temperature sensor 61, whereby a flow rate that is independent of the ambient temperature can be detected.

Otherwise, as a flow rate detector of the differential-pressure type, a differential-pressure type pressure sensor illustrated in FIG. 5B may also be used, in which case a flow rate can also be measured, for example.

(Measurement Device 12)

The measurement device 12 detects a specific gas that is contained in exhalation. As mentioned above, the specific gas is nitrogen monoxide in the present embodiment. As used herein, “specific” refers to an ability to selectively detect some of the various elements and molecules that are contained within exhalation, without having to detect only one specific species. For example, the measurement device 12 may at least have a sensitivity that is low enough to indicate substantially zero sensitivity for nitrogen, oxygen, water, and carbon dioxide that are contained in large amounts in exhalation, while being able to detect nitrogen monoxide. For example, even if the measurement device 12 is able to detect a molecule other than nitrogen monoxide that is hardly contained in exhalation, it suffices if that molecule is substantially not contained in exhalation.

As the measurement device 12, for example, sensors based on detection principles such as the electrochemical type, the optical type, the semiconductor type, etc., can be used. Specifically, electrons occurring when nitrogen monoxide molecules that are diffused within the sensor undergo an oxidation-reduction reaction with a catalyst may be detected with electrodes in the sensor, and based on the detected amount of electric current, a concentration of nitrogen monoxide can be measured.

### [Operation of the Exhalation Measurement Device 1]

Next, with reference to FIG. 2 and FIG. 9, an operation of the exhalation measurement device will be described. FIG. 9 is a flowchart illustrating an operation of the exhalation measurement device. The measurement can be generally classified into: steps of generating exhalation and temporarily stocking the exhalation in the chamber 8 (S1 to S8); and steps of measuring a concentration of the specific gas within the exhalation retained in the chamber 8 (S9 to S14).

(Step of Stocking Exhalation)

First, the subject or operator turns ON the power switch 15 of the exhalation measurement device 1, and presses a button to begin measurement from the input device 13, thus beginning measurement. Instead of beginning measurement with an input via the input device 13, a change in pressure that is associated with the subject generating exhalation may be detected by the pressure sensor 6, thus beginning measurement. Thereafter, in accordance with the procedure in the flowchart shown in FIG. 9, the control circuit 50 controls the respective component elements of the exhalation measurement device 1. When beginning measurement, the valve 56 provided in the first path 54 of the gas transporter 18 is closed so that exhalation will not move from the chamber 8 toward the pump 11.

The subject holds the handle section 2 in a hand, places the orifice 5 against his or her mouth, and breathes in. As a result of this, the lungs of the subject are filled with the air around the exhalation measurement device 1, which has passed through the second filter 34 and from which nitrogen monoxide has been removed (S1). Next, the subject breathes out, so as to blow exhalation into the handle section 2 through the aperture. Via the handle tube 3, the exhalation is introduced into the introduction path 51 of the main body 4 (S2).

The pressure sensor 6 detects a change in pressure in the introduction path 51, and the control circuit 50 moves the adjustment valve 52 of the flow rate adjuster 7. As a result, the exhalation is transported to the chamber 8 through the introduction path 51 (S3). Moreover, the valve 56 provided in the first path 54 of the gas transporter 18 opens.

While the exhalation is being blown in, the pressure of the exhalation is measured by the pressure sensor (S4), and based on the result of measurement, the control circuit 50 moves the adjustment valve 52 of the flow rate adjuster 7 in the introduction path 51, whereby the flow rate of the exhalation moving in the introduction path 51 is adjusted (S5).

The control circuit measures an elapsed time since the pressure sensor 6 detected a change in pressure, and pressure measurement (S4) and adjustment of the flow rate of the exhalation (S5) are performed until a predetermined time elapses (S6).

After the lapse of the predetermined time, the control circuit 50 determines whether the pressure is within a predetermined range, and whether the flow rate is within a predetermined range (S7). If the pressure and the flow rate are within the predetermined ranges (S7), the control circuit 50 closes the adjustment valve 52 of the flow rate adjuster 7, thus ending stocking of the exhalation in the chamber 8 (S8). The control circuit 50 causes the display device 14 to display information indicating that exhalation has been properly acquired. With this, the subject ends blowing of breath, and disengages the handle section 2 from his or her mouth.

If the pressure and the flow rate are outside the predetermined ranges (S7), the control circuit 50 causes the display device 14 to display information indicating failure of measurement (S15). Also, information to prompt another measurement is displayed. With this, the subject or operator carries out another measurement (S1 to S8)).

In the above processes, the air that existed in the introduction path 51 prior to beginning the measurement first flows into the chamber 8. Thereafter, as the exhalation is introduced into the chamber 8, the air that first flowed into the chamber 8 is discharged through the discharge port of the chamber 8. With such operation, in the case where the blowing time of exhalation is set to about 30 seconds, for example, the exhalation which is obtained in the last few seconds in the blowing time of exhalation will be retained in the chamber 8.

(Measurement Step)

The control circuit 50 drives the pump 11 of the gas transporter 18 to transport the exhalation in the chamber 8 to the measurement device 12 (S9). At this point, a flow rate is detected by using the flow rate detector 10, and based on the result of detection, the pump 11 is controlled to attain a predetermined flow rate, thereby adjusting the flow rate of exhalation being introduced into the measurement device 12. The control circuit 50 drives the pump 11 for a certain period of time. Thus, the measurement device 12 detects the flow rate of nitrogen monoxide in the exhalation which comes transported by the pump (S10).

When measurement of a predetermined amount of exhalation is completed, the control circuit 50 controls the switching device 9 of the gas transporter 18 so as to connect the second path 55 that connects to the reference gas generator 17 and the third path 59 that connects to the pump 11 (S11), and controls the pump 11 so that the reference gas will be introduced into the measurement device 12 similarly to exhalation. The pump 11 sucks the reference gas that is generated via passage through the reference gas generator 17, and transports it to the measurement device 12 (S12). The measurement device 12 detects a concentration of nitrogen monoxide in the reference gas that comes transported by the pump (S13).

After the measurement is completed, the control circuit 50 corrects the result of measurement of concentration of nitrogen monoxide in the exhalation by using a result of measurement of concentration of nitrogen monoxide in the reference gas, and displays it on the display device as the result of measurement (S14). Thus, exhalation measurement by the exhalation measurement device 1 is ended. The concentration of nitrogen monoxide within exhalation is, generally speaking, a concentration of several ppb to 50 ppb, for example.

With the exhalation measurement device 1 according to the present embodiment, since the supply tube 29 is connected to the reference gas generator 17, even if the exhalation measurement device 1 is stored under varying environments in a medical institution, deterioration in the adsorption ability of the first filter of the reference gas generator 17 is suppressed. This will be described with reference to experimental results.

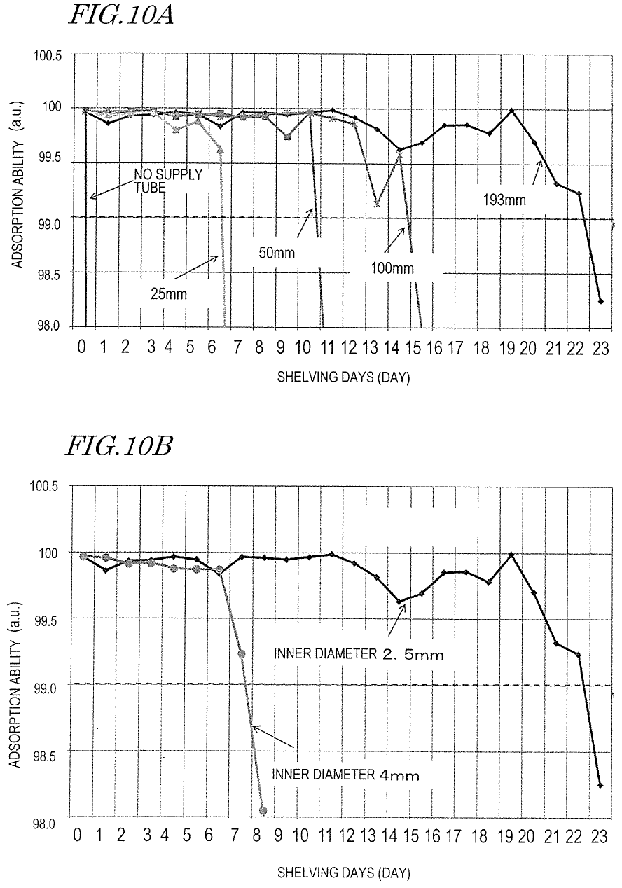

FIG. 10A shows results of examining deteriorations in performance of the first filter, where polytetrafluoroethylene tubes with an inner diameter of 2.5 mm, having respective lengths of 25 mm, 50 mm, 100 mm, and 193 mm, were prepared and used as the supply tube 29 of the reference gas generator 17.

The first filter has a diameter of 12, and a length of 32 mm. The reference gas generator 17 was stored in an environment containing 4% alcohol, and its adsorption ability was regularly measured. The horizontal axis represents the number of storing days, and the vertical axis represents an adsorption ability against the nitrogen monoxide adsorption ability of the reference gas generator 17 before storage, which is defined as 100. For comparison, a result of a reference gas generator which was not connected to the supply tube is also shown. FIG. 10B shows experimental results following a similar procedure, where the supply tubes 29 had respective inner diameters of 4 mm and 2.5 mm. The supply tubes 29 all had a length of 193 mm.

As shown in FIG. 10A, in the absence of the supply tube 29, the adsorption ability lowers to 98% or less immediately after start of the experiment. On the other hand, when the supply tube is 25 mm long, an adsorption ability of 99% or more is maintained until 6 days have passed. With the supply tubes 29 that are 50 mm, 100 mm, and 193 mm long, an adsorption ability of 99% or more is maintained up to 10 days, 14 days, and 22 days, respectively. Thus, it is indicated that, as the supply tube becomes longer, the adsorption ability is maintained for longer periods.

Moreover, as shown in FIG. 10B, it can be seen that the period during which high adsorption ability can be maintained becomes longer as the inner diameter of the supply tube 29 becomes smaller. In the cases where the inner diameter is 4 mm and 2.5 mm, respectively, an adsorption ability of 99% or more is maintained up to 7 days and 22 days.

The reason why providing the supply tube allows the nitrogen monoxide adsorption ability of the first filter to be maintained for a long period of time is unclear as yet. According to a study by the inventors, alcohol molecules that have arrived at the first filter 23 are adsorbed by the first filter 23, and therefore, when the supply tube 29 is stored in an alcohol-containing environment, the alcohol concentration near the first aperture 23a of the first filter 23 is considered to always have a near-zero value. On the other hand, the third aperture 29a of the supply tube 29 is in contact with the environmental ambient. As a result, the difference in alcohol concentration between the third aperture 29a of the supply tube 29 and the first aperture 23a of the first filter 23 is always constant, and alcohol's arrival at the first filter 23 is governed by a diffusion that is based on a concentration gradient, such that the amount of diffused alcohol per unit area remains constant irrespective of lapse of time. Presumably for this reason, the number of alcohol molecules arriving at the first filter 23 decreases as the length of the supply tube 29 becomes longer, and as the cross-sectional area (inner diameter) becomes smaller.

Based on these results, in the exhalation measurement device according to the present embodiment, the performance of the reference gas generator 17 is less likely to deteriorate even in an environment containing alcohol or the like, and the reference gas generator 17 is able to generate for long periods of time a reference gas from which a specific gas has been adequately removed. Thus, a highly accurate measurement is possible by using the reference gas for measuring the specific gas within exhalation, and the highly accurate measurement can be performed stably for long periods of time. Although alcohol is used in the experimental example, the exhalation measurement device according to the present disclosure is considered to provide similar effects also for any compounds other than alcohol that may degrade the adsorption ability of the first filter 23.

[Other Implementations]

The exhalation measurement device according to the present disclosure admits of various modifications. For example, as the supply tube 29 of the reference gas generator, from the standpoint of flexibility for facilitating installation within the space of the main body 4, a resin supply tube would be suitable. However, when previous designing of the placement of the supply tube 29 within the main body 4 is possible, a hard resin tube, a metal tube, or a tube including overlaid resin moldings, etc., may be used to form the supply tube. If the supply tube is composed of a metal such as iron or copper, any gaseous body that tries to permeate into the supply tube from the metal tubular body can be blocked. In the case of adopting a supply tube made of any such hard material, a construction in which the case 19 of the reference gas generator 17 and the supply tube 29 are integrally formed may be adopted.

Although the embodiment illustrates the exhalation measurement device with respect to an example of detecting nitrogen monoxide within exhalation, the exhalation measurement device may also measure the concentration of any other gas besides nitrogen monoxide. For example, carbon monoxide, aldehydes, or the like may be measured. In this case, the exhalation measurement device would include a measurement device 12 which is suitable for the measurement of carbon monoxide, aldehydes, etc., and a first filter and a second filter containing an adsorbent that adsorbs carbon monoxide, aldehydes, etc.

### INDUSTRIAL APPLICABILITY

The present disclosure can be utilized for exhalation measurement devices to be used in e.g. detecting asthma or checking pulmonary function.

### REFERENCE SIGNS LIST

1 exhalation measurement device
2 handle section
3 handle tube
4 main body
5 orifice
6 pressure sensor
7 flow rate adjuster
8 chamber
9 switching device
10 flow rate detector
11 pump
12 measurement device
13 input device
14 display device
15 power switch
16 memory
17 reference gas generator
18 gas transporter
19 case
20 intake port
21 exhaust port
22 aeration path
23 first filter
23a first aperture
24 gasket
24a first aperture
25 gasket
25a second aperture
26 adsorbent
27 check valve
28 attachment section
29 supply tube
29a third aperture
29b fourth aperture
31 sub-housing
32 inhalation path
33 inhalation path
34 second filter
34a aperture
34b adsorbent
34c aperture
35A, 35B one-way valve
36 suction hole
37 discharge hole
41 main housing
41a external air hole
50 control circuit
51 introduction path
51a adjustment hole
51b port
51c port
52 adjustment valve
52a tapered portion
53 driving device
54 first path
55 second path
56 valve
57 valve
58 driving device
59 third path
60 thermal-type flow rate detector
61 ambient temperature sensor
62 upstream temperature sensor
63 heater
64 downstream temperature sensor
65 substrate
65r recess

1. An exhalation measurement device comprising:
a chamber to retain exhalation;
a reference gas generator to generate a reference gas;
and
a measurement device to measure concentrations, respectively, of exhalation being retained in the chamber and a specific gas within the reference gas,
the reference gas generator including:
a case having an intake port and an exhaust port, and an aeration path connecting the intake port and the exhaust port;
a first filter having a first aperture and a second aperture respectively on the intake port side and the exhaust port side in the aeration path within the case, the first filter being disposed in the aeration path to adsorb the specific gas; and
a supply tube having a third aperture and a fourth aperture, the fourth aperture being connected to the intake port of the case, wherein
a distance from the third aperture of the supply tube to the first aperture of the first filter is longer than a distance between the first aperture and the second aperture of the first filter.

2. The exhalation measurement device of claim 1, wherein a distance between the third aperture and the fourth aperture of the supply tube is longer than the distance between the first aperture and the second aperture of the first filter.

3. The exhalation measurement device of claim 1, wherein the distance between the third aperture and the fourth aperture of the supply tube is 50 mm or more.

4. The exhalation measurement device of claim 1, wherein the distance between the third aperture and the fourth aperture of the supply tube is 100 mm or more.

5. The exhalation measurement device of claim 1, wherein a channel of the supply tube has a cross section which is smaller than a cross section of the first aperture that is perpendicular to a straight line connecting the first filter and the second aperture.

6. The exhalation measurement device of claim 1, wherein the supply tube has an inner diameter of 5 mm or less.

7. The exhalation measurement device of claim 1, wherein the supply tube is composed of a resin.

8. The exhalation measurement device of claim 7, wherein the resin is a fluoroplastic.

9. The exhalation measurement device of claim 1, wherein the reference gas generator includes a check valve disposed between the intake port of the case and the first aperture.

10. The exhalation measurement device of claim 1, comprising:
a gas transporter including a pump to selectively transport the exhalation being retained in the chamber or the reference gas generated by the reference gas generator to the measurement device; and
a control circuit to control an operation of the gas transporter.

11. The exhalation measurement device of claim 10, further comprising an exhalation generation unit including:
an aperture through which exhalation and inhalation pass,
an inhalation path having a suction hole and connecting the aperture with the suction hole,
a second filter to adsorb the specific gas, the second filter being located in the inhalation path,
an exhalation path having a discharge hole and connecting the aperture with the discharge hole, wherein,
exhalation which is sent from the discharge hole is introduced into the chamber.

12. The exhalation measurement device of claim 11, further comprising a main housing which accommodates the chamber, the reference gas generator, the measurement device, the gas transporter, and the control circuit, wherein the exhalation generation unit is mounted to the main housing.

13. The exhalation measurement device of claim 11, further comprising a handle section including a sub-housing which is grippable by a subject, wherein
the exhalation generation unit is accommodated in the sub-housing.

14. The exhalation measurement device of claim 10, further comprising:
an introduction path having an inlet through which the exhalation is introduced, the introduction path being connected to the chamber;
a flow rate adjuster to adjust a flow rate of the exhalation flowing in the introduction path; and
a pressure sensor to measure a pressure in the introduction path, wherein,
the control circuit controls the flow rate adjuster based on an output from the pressure sensor.

15. The exhalation measurement device of claim 10, wherein,
the gas transporter further includes
a switching device to switch between a first path connected to the chamber and a second path connected to the pump, and allow the first path or the second path to connect to a third path that is connected to the pump, and
a flow rate detector to measure a flow rate of the exhalation or the reference gas flowing in the third path; and
the control circuit controls the switching device, and controls the pump based on an output from the flow rate detector.
